(19) United States
(12) Patent Application Publication
(10) Pub. No.: US 2022/0160456 A1
(43) Pub. Date: May 26, 2022

(54) IMAGING MARKER AND METHOD

(71) Applicant: Beekley Corporation, Bristol, CT (US)

(72) Inventors: Mark Broyles, Plainville, CT (US); Yuanxiong Luo, Farmington, CT (US); Allison Ostrout, Statesboro, GA (US); Maeghan E. Traboulsi, Fairfax, VA (US)

(21) Appl. No.: 17/456,516

(22) Filed: Nov. 24, 2021

Related U.S. Application Data
(60) Provisional application No. 63/117,783, filed on Nov. 24, 2020.

(51) Int. Cl. A61B 90/00

(52) U.S. Cl. CPC A61B 90/39; A61B 2090/3966; A61B 2090/3991

(57) ABSTRACT

An imaging marker comprises a flexible, axially-elongated line-shaped marker, an underlying adhesive, and a flexible, axially-elongated foam spacer extending between an underside of the line-shaped marker and the adhesive. The line-shaped marker is partially radiopaque, partially radiolucent, or is radiopaque, and the foam spacer and adhesive are radiolucent at the levels of radiation used during an imaging procedure. The marker and foam spacer flex and substantially conform to a curvilinear contour of the skin, follow the curvilinear contour of a scar or other anatomical feature on the skin, and prevent forces exerted thereon in flexibly conforming to and following the curvilinear counters from detaching the adhesive from the skin during the imaging procedure.

### CROSS-REFERENCE TO PRIORITY APPLICATION

This patent application claims priority under 35 U.S.C. § 119 to U.S. provisional patent application No. 63/117,783, filed Nov. 24, 2020, entitled “Imaging Marker and Method,” which is hereby expressly incorporated by reference in its entirety as part of the present disclosure.

### FIELD OF THE INVENTION

The present invention relates to imaging markers, such as for x-ray, computerized tomography (“CT”), or tomosynthesis imaging, and more particularly, to medical imaging markers.

### BACKGROUND INFORMATION

Medical imaging scar markers are used during x-ray or CT imaging, such as during 2D mammography or 3D mammography, to mark the locations of scars and otherwise reduce or eliminate confusion caused by architectural distortion in images. Scar markers act as a reference point to identify the location(s) of previous surgeries and can correlate architectural distortion from previous biopsy or surgical sites by delineating the exact locations of prior incisions. With the advent of digital breast tomosynthesis (“DBT”), radiologists are seeing more architectural distortion from older benign surgeries than they had previously in full-field digital mammography (“FFDM”), necessitating the need to look back at years of previous images to determine if the architectural distortion on the DBT image is correlated to a previous surgery or is something new. For example, when a breast has been subjected to trauma, such as an excisional biopsy or lumpectomy, it is important that the radiologist see the resulting scar, correlate it with a scar marker in the image, and compare the image to one or more images of the same area in prior years to understand that the image of the scar is dermal and is not a new anatomical feature within the breast. When used year after year to mark surgical sites, scar markers help distinguish between a new or recurring breast cancer and normal post-surgical changes which may exhibit characteristics of an evolving cancer.

The assignee of the present invention manufactures and sells scar markers. Such scar markers include either a low-density, see-through line-shaped marker or a radiopaque line-shaped marker, and are configured to be releasably attached to the patient's skin by an adhesive-backed substrate. Scar markers can be used to mark field borders, tangents, scars, and sarcomas. Such markers are flexible and contour to the skin. Exemplary such scar markers are sold by Beekley Corp. under the brands TomoSPOT™, CT-SPOT™ and S-SPOT™.

Scar markers are intended to clearly communicate the location, length, and shape of a post-surgical scar. When properly positioned and adhered to the skin, they can communicate the location(s) of prior surgical site(s), maintain a reference point to original surgical sites from year to year, and correlate surgical histories with mammographic findings. In addition, calcifications found in the area marked by the scar marker can be examined relative to their location. Accordingly, when properly positioned and adhered to the skin, scar markers can reduce unnecessary additional imaging and diagnostic tests by differentiating suspicious lesions from benign architectural distortion as a result of a benign surgery.

In FIG. 1, an exemplary prior art medical imaging scar marker comprises a self-adhesive label [16] with an extruded imaging material [12]. The marker is shown applied to the skin surface [30]. The marker is used to trace a scar [31] appearing on a patient's skin [30] by aligning the marker to the scar and pressing it onto the skin. The self-adhesive label [16] is die cut from a solid or non-woven film. When using a solid or non-woven film, the label must be thin to minimize attenuation of x-ray energy [32] so that the label [16] does not appear on the x-ray Image [33]. As shown typically in FIG. 1, it is desired to only see the extruded imaging material [12] appear as a mark [34] on the x-ray image [33].

In FIG. 2, the medical imaging marker is shown with the extruded imaging material removed for clarity. FIG. 3 is a partial, magnified view of the marker of FIG. 2. As can be seen, the imaging marker label [16] is designed with diamond-shaped segments [26] with rectangular or square bridges [24] between them. When the marker is required to follow a curve in a scar [31], the label [16] must be shaped around the curve's axis [34]. To maintain a coplanar relation between each of the diamond segments [26], each bridge [24] must bend around an axis [35] local to each bridge [24]. The bend in each bridge requires the bridge edge [38] closest to the scar curve axis [34] to compress while causing the opposite bridge edge [37] to stretch. Because the marker label [16] is a solid or non-woven material, it does not readily stretch or compress. Bending forces applied will cause each bridge [24] to deform in the direction of least resistance which is typically the axis [36] on each bridge [24]. The deformation of each bridge [24] across each axis [36] causes the diamond-shaped segments [26] to move away from their desired coplanar formation, as shown typically in FIG. 2. This can lead to great difficulty in placing the imaging marker flat onto the skin surface [30].

Another drawback of prior art scar markers is edge lifting. In FIG. 4, the cross-section of a solid label [16] of the exemplary prior art skin marker of FIGS. 1-3 is shown on a planar skin surface [30]. As can be seen, on a planar surface the upper surface [39] of the label and lower surface [40] of the label are of equal length. In FIG. 5, the same label [16] is shown applied to a curved skin surface [41] as is often found on anatomical features. Because of the curved nature of the skin surface, the upper surface [39] and lower surface [41] have different radii and therefore must define slightly different lengths. As shown in FIG. 6, one potential result of applying the adhesive-backed solid film label [16] to the curved surface [41] is illustrated. As can be seen, the upper surface [39] of the solid film label is subjected to stretching or tensile forces, and the lower surface [40] of the solid film label is subjected to shortening or compressive forces. Because the prior art label is a solid-film or dense fiber structure, the opposing forces cause the label to attempt to return to its natural planar shape and lift away from the skin, as indicated by the arrow [42] in FIG. 6.

A further drawback of prior art scar markers is wrinkling. As shown in FIG. 7, the above-described exemplary prior art skin marker is shown applied to a skin surface [30] containing a wrinkle [43]. As indicated above, when using a solid or non-woven film, the label must be thin to minimize attenuation of x-ray energy [32] so that the label [16] does not appear on the x-ray image [33]. As shown typically in FIG. 7, it is desired to only see the extruded imaging material [12] appear as a mark [34] on the x-ray image [33]. If the skin surface contains a wrinkle, such as the exemplary wrinkle [43], the thin label material [16] may fold [44] into the wrinkle. FIG. 8 is a cross-sectional view of the label material [16] and wrinkle [43] and illustrates the manner in which a fold [44] of the label material [16] may be received in the wrinkle [43]. The fold [44] in the label [16] can create a mass large enough to attenuate enough x-ray energy to cause the mass to show on the image as an unknown artifact, as illustrated by the exemplary artifact [45] in FIG. 7.

Correct visual communication of a scar marker is important to accurately document a scar from year to year. The scar marker should follow the shape of the scar without movement and remain in place during the imaging procedure. During certain imaging procedures, such as in mammography, the patient's skin or tissue is subject to manual manipulation and compressive and tensile forces experienced in positioning and compressing the breast. One drawback encountered with prior art scar markers is that the manipulation and compressive and tensile forces applied to the breast tissue during mammography can cause prior art scar markers, at times, to pop off on one side, move, or fall off. This problem can be exacerbated when the marker is bent into a curvilinear shape and subjected to deformation, such as the deformation of the bridges [24] across their axes [36] described above, which causes the adhesive-backed label of the marker to move away from its desired coplanar formation with the skin. These drawbacks also can be exacerbated if the marker is subjected to edge lifting and/or wrinkling, as described above. When any or all of these events occur, the scar marker may not accurately follow the shape of the scar, may lead to a miscommunication about a patient's breast to the technologist or radiologist, and may require that an image be repeated.

It is an object of the present invention, and/or of embodiments thereof, to overcome one or more of the above-described drawbacks and/or disadvantages of the prior art.

### SUMMARY OF THE INVENTION

In accordance with a first aspect, the present invention is directed to an imaging marker for use in connection with an imager. The imaging marker comprises a linear-shaped marker that is flexible, defines an underside and an elongated axis, and is visible on an image of the marker taken by the imager in connection with an imaging procedure. The imaging marker further comprises an adhesive and a foam spacer that is located between the adhesive and the marker and is translucent or substantially invisible on the image of the marker taken by the imager in connection with the imaging procedure. The adhesive is configured to releasably attach the imaging marker to a surface of a person's skin undergoing the imaging procedure at an interface of the imaging marker and the skin. The foam spacer defines a thickness between the adhesive and substantially the entirety of the underside of the marker, and spaces substantially the entirety of the underside of the marker away from the skin. The foam spacer defines an axially-elongated portion extending along the elongated axis of the linear marker between the linear marker and the adhesive, and a plurality of laterally-extending portions. A plurality of the laterally-extending portions are located on opposite sides of the elongated axis relative to each other, and at least a portion of a plurality of the laterally-extending portions are axially spaced relative to each other along the elongated axis. The foam spacer is configured to flex at least between the axially-spaced, laterally-extending portions to thereby allow the spacer to flex with the linear-shaped marker.

In some embodiments of the invention, the foam spacer defines a plurality of pairs of laterally-extending portions extending laterally on opposite sides of the elongated axis relative to each other, and relatively narrow-width portions located between axially-spaced pairs of laterally-extending portions.

In some embodiments of the invention, the imager generates images by transmitting radiation, the marker is formed by at least one radiopaque portion that is at least partially radiopaque or partially radiolucent at a level of radiation used by the imager in connection with the imaging procedure, and the spacer is substantially radiolucent at the level of radiation used by the imager in connection with the imaging procedure.

In some embodiments of the invention, the spacer extends between the linear-shaped marker and the adhesive, and defines a thickness between an underside of the linear-shaped marker and the adhesive of at least about ½ millimeter. In some such embodiments, the spacer defines a thickness between the underside of the linear-shaped marker and the adhesive within the range of about ½ millimeter to about 1 millimeter. In some embodiments of the invention, the thickness between the underside of the linear-shaped marker and the adhesive is substantially uniform.

In some embodiments of the invention, the imaging marker is mounted on a releasable liner, and the releasable liner is releasably attached to the adhesive. In some such embodiments, the marker defines a continuous linear shape extending along the elongated axis, and the releasable liner defines an axially-elongated shape extending along the elongated axis of the linear marker. In some embodiments, the linear marker and releasable backing are configured to be torn, cut or separated at desired locations to form individual imaging markers therefrom at desired lengths.

In some embodiments of the invention, the foam is a thermoplastic or thermoset foam. In some embodiments, the foam is a closed-cell foam. In some embodiments, the linear-shaped marker is made of a thermoplastic including a filler of sufficient density to make the marker visible on an image of the marker taken by the imager in connection with the imaging procedure.

In accordance with another aspect, the present invention is directed to imaging marker for use in connection with an imager. The imaging marker comprises first means for releasably attaching the imaging marker to a surface of the skin of a person undergoing the imaging procedure. The surface of the skin defines a curvilinear contour and a scar or other anatomical feature also defining a curvilinear contour. The imaging marker comprises second means visible on an image of the marker taken by the imager in connection with an imaging procedure and forming a line-shaped image thereof in the image. The second means is for flexing and substantially conforming to the curvilinear contour of the skin and for following the curvilinear contour of the scar or other anatomical feature on the skin. Third means located between the first and second means is translucent or substantially invisible on the image of the marker taken by the imager in connection with the imaging procedure. The third means is for spacing substantially the entirety of the underside of the first means away from the skin, flexing and substantially conforming to the curvilinear contour of the skin, following the curvilinear contour of the scar or other anatomical feature on the skin, and preventing forces exerted thereon in flexibly conforming to and following the curvilinear counters from detaching the first means from the skin.

In some embodiments of the invention, the first means is an adhesive, the second means is an axially-elongated, line-shaped marker, and the third means is a foam spacer. The foam spacer defines an axially-elongated portion extending along the elongated axis of the line-shaped marker between the line-shaped marker and the adhesive. A plurality of laterally-extending portions of the foam spacer are located on opposite sides of the elongated axis relative to each other, and are axially-spaced relative to each other along the elongated axis to thereby define bridges located between laterally-extending portions.

In accordance with another aspect, the present invention is directed to a method comprising:

preparing an imaging marker for attachment to a surface of the skin of a subject to be imaged, wherein the surface of the skin defines a curvilinear contour and a scar or other anatomical feature also defining a curvilinear contour, the imaging marker includes a flexible line-shaped marker portion that is visible on an image of the marker taken by an imager in connection with an imaging procedure, an adhesive, and a foam spacer located between the line-shaped marker portion and the adhesive that is translucent or substantially invisible on the image of the marker taken by the imager in connection with the imaging procedure, and the preparing includes flexing and substantially conforming the line-shaped marker portion to the curvilinear contour of the skin and causing the line-shaped marker portion to substantially follow the curvilinear contour of the scar or other anatomical feature on the skin;

releasably attaching to the surface of the skin the imaging marker and, in connection with such attaching, substantially conforming the line-shaped marker portion to the curvilinear contour of the skin, and causing the line-shaped marker portion to substantially follow the curvilinear contour of the scar or other anatomical feature on the skin;

imaging with the imager the marker portion of the imaging marker and at least the portion of the person underlying the marker such that the marker portion is visible and the foam spacer is translucent or substantially invisible on the image of the marker taken by the imager; and

during the releasably attaching and imaging of steps, absorbing forces exerted on the imaging marker during the flexible conforming to and following of the curvilinear counters within the foam spacer, and thereby preventing the imaging marker from detaching from the skin during the releasably attaching and imaging of steps.

In some embodiments of the invention, the imaging includes transmitting radiation through the imaging marker at a level at which the marker portion is partially radiopaque, partially radiolucent to the transmitted radiation and the spacer is substantially radiolucent to the transmitted radiation.

One advantage of the present invention, and/or of one or more embodiments thereof, is that the line-shaped marker and foam spacer flex and substantially conform to the curvilinear contours of the skin, follow the curvilinear contours of scars or other anatomical features on the skin, and prevent forces exerted thereon in flexibly conforming to and following the curvilinear counters from detaching the adhesive layer from the skin during the imaging procedure. Accordingly, the imaging markers and method of the invention substantially obviate the problems encountered in connection with the above-described prior art scar markers, such as deformation of the solid film or non-woven label when bending the label to conform to the contour of the skin and/or follow the contour of a scar or other anatomical feature on the skin, and the associated inability of such prior art scar marker labels to be placed flat on the surface of the skin or otherwise maintain a coplanar relation between the label and skin. Yet another advantage is that the problems encountered in connection with the above-descried prior art scar markers with respect to edge lifting and wrinkling can be substantially avoided. Accordingly, the imaging markers and method of the invention facilitate the proper positioning of the markers and their adhesion to the skin. Yet another advantage of the imaging markers and method of the invention is that the foam spacer or substrate can absorb the compressive and tensile forces exerted on the marker during manipulation and compression of breast tissue during mammography procedures, and thereby avoid the problems encountered with the above-described prior art scar markers of popping off on one side, moving or falling off during mammographic procedures. Accordingly, the imaging markers and method of the invention can obviate the problems encountered with the above-described prior art scar markers where the scar marker may not accurately follow the shape of the scar, may lead to miscommunication about a patient's breast to the technologist or radiologist, and may require repeated imaging.

Other advantages of the present invention, and/or of the embodiments thereof, will become more readily apparent in view of the followed detailed description of embodiments of the invention and accompanying drawings.

### BRIEF DESCRIPTION OF THE DRAWINGS

FIG. 1 is a perspective view of a prior art scar marker applied to a substantially flat skin surface where the adhesive-backed label of the scar marker has a substantially coplanar formation with the underlying skin, and an exemplary x-ray image of the scar marker on an opposite side of the underlying skin relative to the scar marker;

FIG. 2 is a perspective view of the prior art scar marker of FIG. 1 with the extruded imaging material removed therefrom for clarity;

FIG. 3 is a magnified, partial perspective view of the label of the prior art scar marker of FIG. 2;

FIG. 4 is a somewhat schematic, partial, cross-sectional view of the prior art solid label of the scar marker of FIG. 1 applied to a substantially planar skin surface;

FIG. 5 is a somewhat schematic, partial, cross-sectional view of the prior art solid scar marker label of FIG. 4 applied to a curved skin surface;

FIG. 6 is a somewhat schematic, partial, cross-sectional view of the prior art solid scar marker label applied to the curved skin surface of FIG. 5 showing an exemplary lifting of the edge of the label away from the curved skin surface;

FIG. 7 is a perspective view of a prior art scar marker applied to a flat skin surface including a wrinkle, and an exemplary x-ray image of the scar marker on an opposite side of the underlying skin relative to the scar marker;

FIG. 8 is a somewhat schematic, partial, cross-sectional view of the prior art scar marker of FIG. 7 showing the label material folded into the wrinkle;

FIG. 9 is a partial, perspective view of linear-shaped or scar marker embodying the present invention;

FIG. 10 is a top plan view of the linear-shaped or scar marker of FIG. 9;

FIG. 11 is a cross-sectional view of the linear-shaped or scar marker of FIG. 9 taken along line A-A of FIG. 10;

FIG. 12 is a partial, perspective view of the linear marker of FIG. 9 removed from its releasable backing and applied to a patient's skin;

FIG. 13 is a perspective view of the scar marker of FIG. 9 with the extruded imaging material or marker removed therefrom for clarity;

FIG. 14 is a partial, magnified perspective view of the foam substrate of the scar marker of FIG. 13;

FIG. 15 is a somewhat schematic, partial, cross-sectional view of the foam substrate of the scar marker of FIGS. 10-12 applied to a curved skin surface; and

FIG. 16 is a somewhat schematic, partial, cross-sectional view of the foam substrate of the scar marker of FIGS. 10-12 applied to a skin surface including a wrinkle.

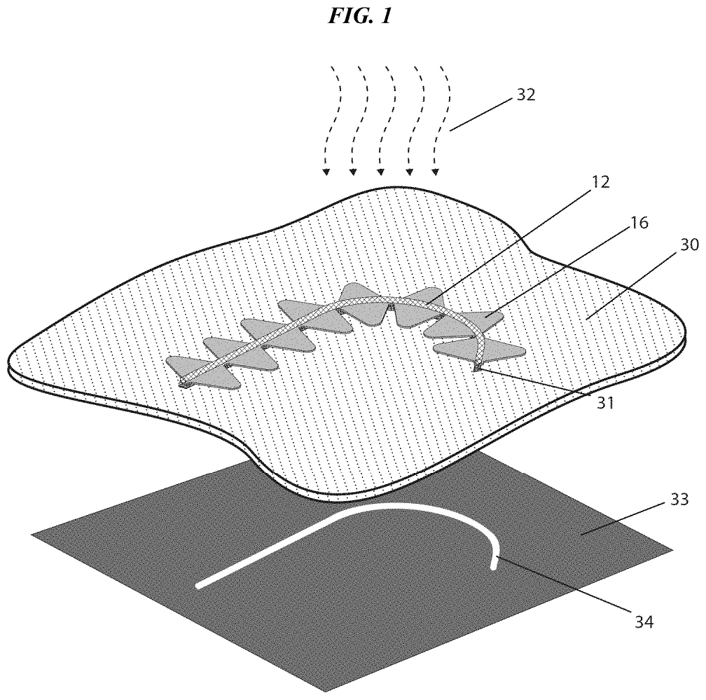

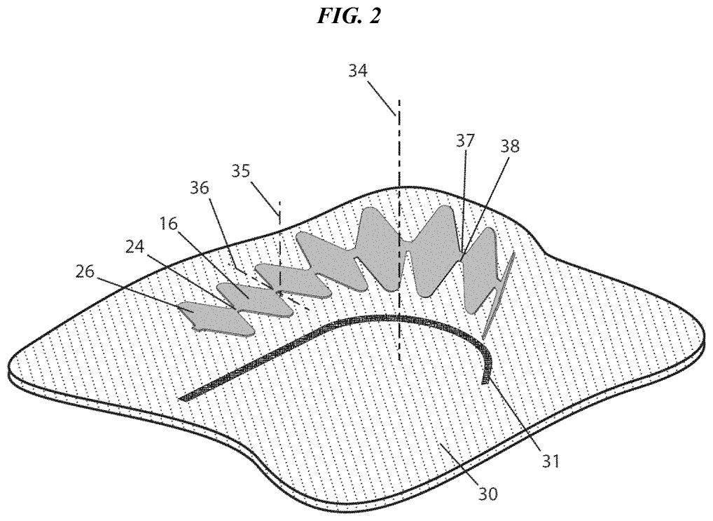

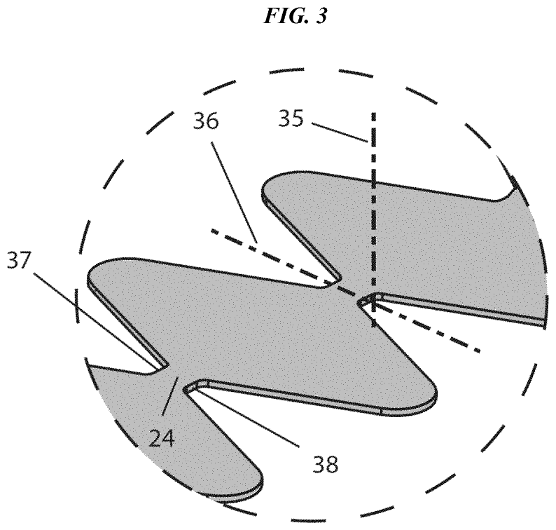

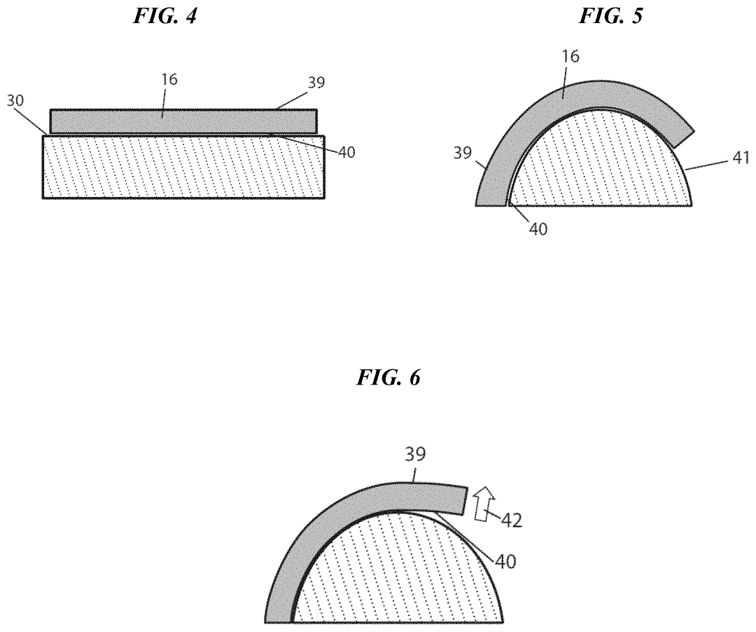

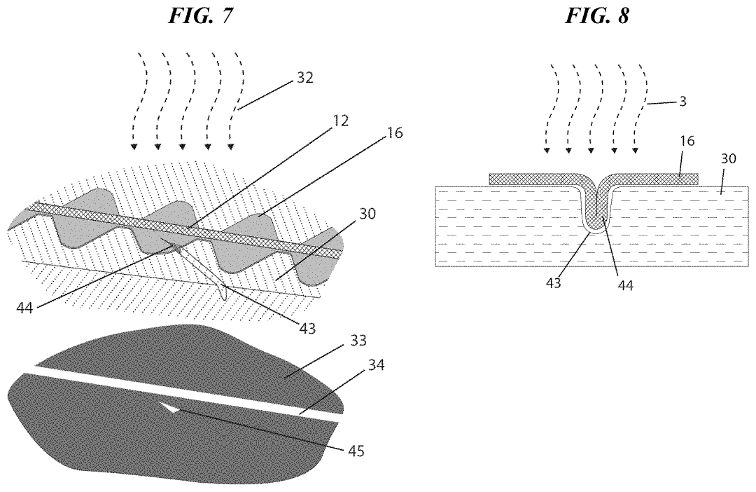

### DETAILED DESCRIPTION OF EMBODIMENTS OF THE INVENTION

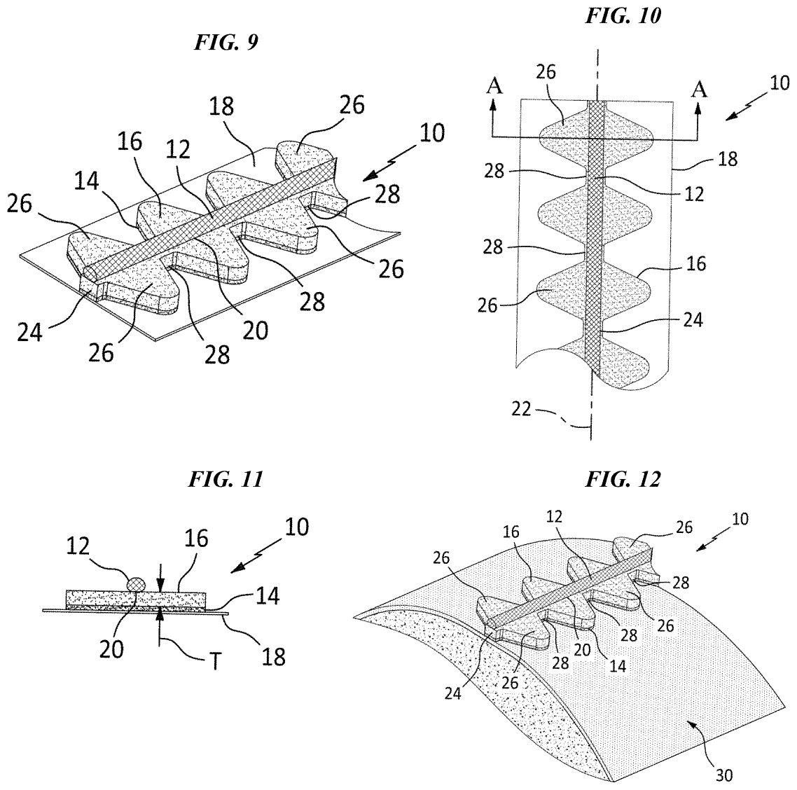

In FIGS. 9-16, an imaging marker embodying the present invention is indicated generally by the reference numeral 10. The imaging marker 10 comprises a marker 12, an adhesive layer or coating 14, and a spacer or substrate 16 located between the adhesive and the radiopaque marker. Preferably, the marker 12 is partially radiopaque and partially radiolucent at the levels of radiation used during the imaging procedures with which the imaging marker 10 is used so that underlying anatomical detail is visible within the image of the marker. On the other hand, the spacer or substrate 16 and underlying adhesive 14 are preferably radiolucent or substantially radiolucent at the levels of radiation used during the imaging procedures with which the imaging marker 10 is used so that the spacer and adhesive are not visible or substantially invisible in the images of the marker. Accordingly, the marker 12 is visible in an x-ray, CT, or other type of image of the marker, whereas the radiolucent spacer 16 and adhesive 14 are translucent or invisible on the image. However, as may be recognized by those of ordinary skill in the pertinent art based on the teachings herein, the marker 12, spacer 16, and adhesive 14 may define any desired densities or imaging characteristics that are currently known, or that later become known, or otherwise as may be desired.

As shown in FIGS. 9-10, the adhesive 14 releasably attaches the imaging marker 10 to a releasable liner 18. As shown typically in FIG. 12, the adhesive layer 14 is configured to releasably attach the imaging marker 10 to a surface of a patient's skin at an interface of the imaging marker and the skin. In the illustrated embodiment, the adhesive 14 is in the form of a coating that underlies the radiolucent spacer 16. The adhesive coating 14 is preferably pressure sensitive in that it releasably attaches the imaging marker to a person's skin when slight pressure is applied thereto. In one embodiment of the present invention, the adhesive 14 is a pressure sensitive acrylic adhesive, and the releasable liner is a silicone treated, polyethylene coated, bleached kraft paper. The adhesive may take the form of any of numerous different types of adhesives or other substances that are currently known, or that later become known for releasably attaching an imaging marker to a person's skin.

The marker 12 defines an underside 20, and the radiolucent spacer 16 defines a thickness “T” between the adhesive 14 and substantially the entirety of the underside 20 of the marker. In the illustrated embodiment, the thickness T is preferably within the range of about ½ millimeter to about 1 millimeter, and is more preferably about 8/10 millimeter. Accordingly, in the illustrated embodiment, the radiolucent spacer 16 spaces substantially the entirety of the underside 20 of the marker 12 away from the skin. On the other hand, if the thickness T of the radiolucent spacer is too great, it could interfere with the ability to handle the marker. For example, if the thickness T is too great, it may reduce the ease of handling the imaging marker and/or interfere with the ability to bend or otherwise deform the imaging marker into a required or desired shape. In addition, when a line or scar marker is used for mammography procedures, such as 2D or 3D mammography, if the thickness T is too great, the imaging marker may create an obstruction or otherwise interfere with the manipulation and/or compression of the breast, may be accidentally pulled, pushed or impacted during the procedure, and as a result, moved out of position or detached from the skin. In addition, if the thickness T of a radiolucent spacer is too great, it may prevent packaging a required or desired number of imaging markers into a conventional or commonly-sized package. Accordingly, in some embodiments, the radiolucent spacer defines thickness T that is sufficient to perform the foregoing function, but nevertheless defines a relatively low profile to facilitate handling, packaging, or otherwise prevent the marker from creating an undesirable obstruction or profile during use. As shown in FIGS. 9-11 and 12, in the illustrated embodiments, the thickness T of the radiolucent spacer is substantially uniform throughout in its normal state when not subjected, for example, to the forces that may be encountered during mammography.

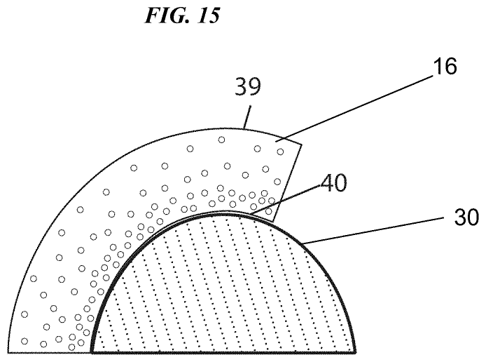

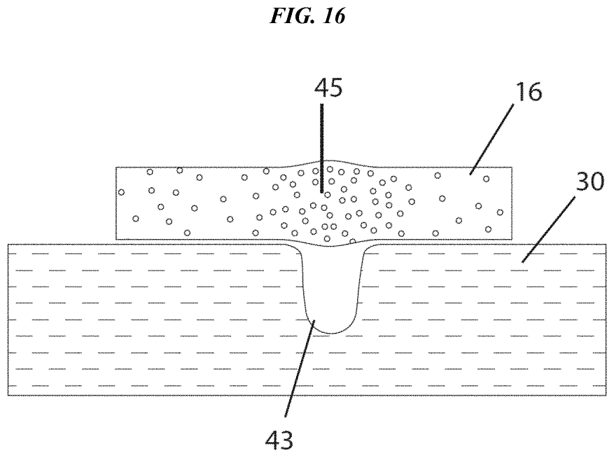

As shown typically in FIGS. 9-12, the marker 12 is linear shaped and defines an elongated axis 22. As shown in FIGS. 9, 10 and 12, the radiolucent spacer 16 defines an axially-elongated portion 24 that extends along the elongated axis 22 between the linear radiopaque marker 12 and adhesive-backed substrate 14. The radiolucent spacer 16 defines a plurality of pairs of laterally-extending portions 26, 26 that extend laterally on opposite sides of the elongated axis 22 relative to each other, and relatively narrow-width portions 28, 28 located between axially-spaced pairs of laterally-extending portions. As can be seen, the laterally-extending portions 26, 26 are axially spaced relative to each other along the elongated axis by the relatively narrow-width portions 28, 28. In the illustrated embodiment, both the radiopaque marker 12 and radiolucent spacer 16 are flexible in order to allow the marker to conform its shape to the contour of the patient's skin to which it is releasably attached and allow it to otherwise take any desired shape. The relatively narrow-width portions 28, 28 located between the laterally-extending pairs 26, 26 allow the laterally-extending pairs to flex relative to each other about the elongated axis 22 in order to flex or deform the shape of the marker as needed. For example, the linear shape of the imaging marker 10 may be flexed and/or deformed as needed in order to mark field borders, tangents, scars, or sarcomas and/or to otherwise clearly denote an area of concern in imaging without lifting or coming off the skin. The radiolucent spacer 16 is sufficiently flexible and/or moldable to form desired shapes as dictated by the anatomy or anatomical features of each patient and, as indicated above, defines a thickness T that is preferably substantially uniform throughout. In the illustrated embodiment, the radiolucent spacer 16 is formed of a thermoplastic or thermoset foam, such as a closed-cell polyethylene foam. In one embodiment, the radiolucent spacer is formed of a polyethylene closed-cell foam. In another embodiment, the radiolucent spacer is formed of a polyethylene open-cell foam. As shown typically in FIGS. 15 and 16, the foam spacer defines a plurality of cells, pockets, or voids throughout. In a closed-cell foam spacer, the cells, pockets, or voids are enclosed within the spacer. In an open-cell foam spacer, on the other hand, the cells, pockets, or voids are open. The cells, pockets, or voids are empty and thus facilitate the ability of the foam spacer to stretch, compress or otherwise deform when subjected to tensile, compressive, and other forces in order to absorb such forces and prevent them from moving or otherwise detaching the marker from the skin. Preferably, the foam spacer 16 is sufficiently elastic to allow it to stretch and/or compress when subjected to such forces, such as during mammography, to prevent them from moving or otherwise detaching the marker from the skin. In a currently preferred embodiment, the elasticity of the foam spacer 16 is characterized by being bendable or folded up to about 180° without causing or exhibiting visible stress cracks therein. As indicated above, the marker 12 is of sufficient density so that it is partially radiopaque, partially radiolucent at the levels of radiation used during the imaging procedure and is sufficiently flexible and/or moldable to form desired shapes as dictated by the anatomy or anatomical features of each patient. In the illustrated embodiment, the marker 12 is made of an extruded thermoplastic including a filler of sufficient density to render the marker partially radiopaque, partially radiolucent at the levels of radiation used during the imaging procedure, such as a 2D or 3D mammography procedure.

As may be recognized by those of ordinary skill in the pertinent art based on the teachings herein, the marker 12 and the radiolucent spacer 16 may be made of any of numerous different materials that are currently known, or that later become known, for purposes of performing the functions of these elements or components of the imaging marker. For example, the radiolucent spacer may be formed of an open-cell or closed-cell foam made of any of numerous different materials that are currently known or that later become known. In addition, the radiolucent spacer need not be a foam, but rather can be made of another radiolucent material. Similarly, the marker 12 may be non-metallic as described above, may be metallic, or may be a combination of metallic and non-metallic materials. For example, the metallic portion could be formed by a flexible, deformable metal wire, such as a steel wire. If non-metallic, or partially metallic, the body of the marker 12 and its radiopaque or partially radiopaque filler may be made any of numerous different materials that are currently known or that later become known. In addition, the imaging marker is configurable to work with any type of imager, imaging process, or imaging system. When configured for each such imager, imaging process, or system, the marker is configured to be partially radiopaque and partially radiolucent so that the marker is visible on an image of the marker taken by such imager, process, or system, but the density of the marker is sufficiently low such that the marker is see-through to allow anatomical detail within the tissue underlying the marker to be visible through the marker within the image and/or shadow of the marker. The spacer, on the other hand, is configured to be translucent or invisible on the image. As also indicated above, in other embodiments, the marker is radiopaque or substantially radiopaque on an image of the marker taken by such imager, process, or system. For example, if configured for use with magnetic resonance (“MR”) imaging (also referred to as MRI or NMR imaging), the marker 12 is made of or includes a material that is partially opaque or partially visible, or is opaque or visible, on an MR image, whereas the spacer 16 is made of or includes a material that is not visible or is translucent on the MR image. Accordingly, the imaging markers can be used with any of numerous different imagers, imaging processes or imaging systems, that are currently known, or that later become known, and may be made of any of numerous different materials required by such imagers, processes or systems in order to cause the marker or marker portion to be opaque or visible in images of the marker, and the spacer to be translucent or invisible in such images.

In the illustrated embodiment, the laterally-extending pairs 26, 26 take the form of tabs, where the tabs are approximately triangular-shaped and the pairs of opposing tabs are approximately diamond shaped. However, as may be recognized by those of ordinary skill in the pertinent art based on the teachings herein, the laterally-extending portions may take any of numerous other shapes, such as semi-circular or semi-oval shapes, and the laterally-extending tabs or other portions need not be laterally aligned, as shown, but may be axially offset relative to each other.

The radiolucent spacer 16 and its underlying adhesive 14 allow the marker to conform to the contour of the patient's skin and otherwise flex or deform into a desired shape in order to releasably attach the marker to the patient's skin in the desired shape without coming off of the skin during an imaging procedure, such as a 2D or 3D mammography procedure. As shown in FIGS. 9-11, the imaging marker 10 is supplied on a releasable backing 18 in strip form that allows the imaging marker to be removed therefrom and, in turn, adhesively attached to a patient's skin. In use, the imaging marker 10 is cut to the desired length with, for example, a pair of scissors (not shown). A pair of scissors or other desired cutting instrument may be used to easily cut through the releasable backing 18 and the imaging marker 10 in order to cut the desired length of imaging marker for a respective application. The imaging marker 10 can be supplied on its releasable backing 18 in a roll form (not shown) and dispensed and cut, torn or otherwise separated to desired lengths as needed while preserving the remainder of the roll for future use.

As shown typically in FIGS. 12-16, the imaging marker 10 is cut to the desired length, is removed from its piece of releasable backing 18, is deformed or otherwise molded into a desired shape, as needed, and is releasably attached to the patient's skin 30 with the adhesive 14. The flexible nature of the marker 10 and adhesive-backed radiolucent spacer 16 allow the marker to conform to the contour of the patient's skin 30 and otherwise conform to or mark the anatomical features of interest. As indicated above, the marker 12 is partially radiopaque, partially radiolucent at the levels of radiation used during the imaging procedure, whereas the radiolucent spacer 16 and adhesive backing 14 are substantially radiolucent at the levels of radiation used during the imaging procedure. As a result, only the marker 12 is visible on a CT or other image taken during the imaging procedure. In addition, the radiolucent spacer 16 spaces substantially the entirety of the underside of the marker 12 away from an interface of the imaging marker and the surface of the skin 30 of the patient.

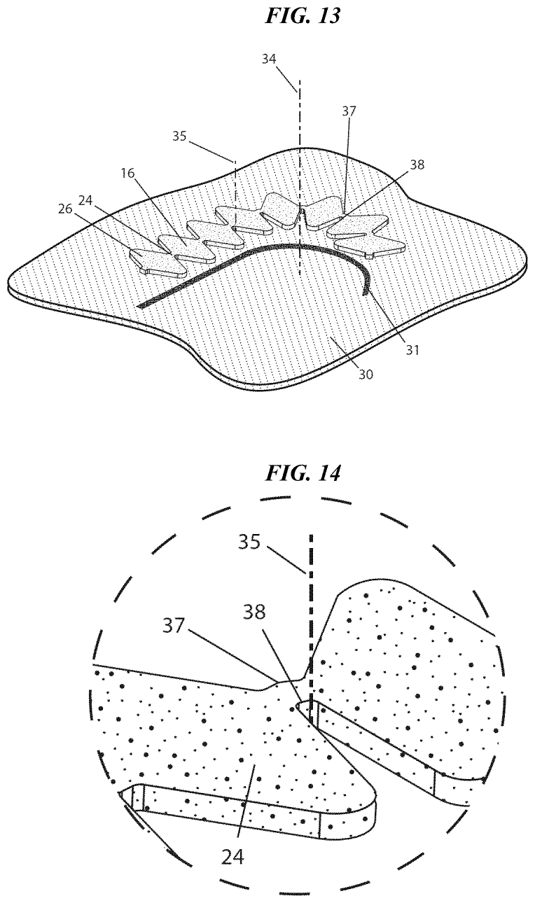

As shown typically in FIGS. 13 and 14, the laterally-extending portions 26, 26 of the spacer or substrate 16 are defined by substantially diamond-shaped segments or pads, and the axially-elongated portion 24 defines a plurality of rectangular or square bridges between the diamond-shaped segments or pads. When the marker is required to follow a curve in a scar 31 on the skin 30, the spacer or substrate 16 must be shaped around the curve's axis 34. To maintain a coplanar relation between each of the diamond-shaped segments 26, each bridge 24 must bend around an axis 35 local to each bridge 24. The bend in each bridge 24 requires the bridge edge 38 closest to the scar curve axis 34 to compress and the opposite bridge edge 37 to stretch. One advantage of this embodiment of the invention is that the spacer or substrate 16 is made of foam, and therefore is able to stretch and compress. The bending force applied to cause the scar marker 10 to conform to the shape of a scar 31 will cause each bridge 24 to deform in or about the respective axis 35 of each bridge 24. The deformation of each bridge 24 across its respective axis 35 causes the diamond-shaped segments 26 to remain in their desired coplanar formation with the skin, which allows ease in placing the imaging marker flat onto and/or into conformity with the surface of the skin 30.

Turning to FIG. 15, the benefit of using a compressible foam for the spacer or substrate 16 with respect to overcoming the problem of edge lifting encountered in the above-described prior art is illustrated. As can be seen, the upper surface 39 of the foam spacer or substrate 16 is able to stretch, and the lower surface 40 of the foam spacer or substrate is able to compress. As a result, the foam 16 is allowed to compensate for the difference in upper and lower surface radii and/or lengths, resist lifting from the skin 30, and obviate the problem of edge lifting encountered in the prior art.

Similarly, as shown in FIG. 16, when applied to a skin surface 30 with one or more wrinkles 43, the foam material of the spacer or substrate 16 is allowed to compress in an area 45 proximate the wrinkle to thereby avoid creating a fold that could be received in the wrinkle. Rather, the foam spacer 16 remains above and substantially outside the wrinkle, and does not create a fold that could be received within the wrinkle 43, or otherwise give rise to the associated undesirable imaging artifacts as encountered in the above-described prior art. The compressible foam allows the spacer or substrate 16 to be made much thicker than the prior art solid or nonwoven labels. The thicker substrate is much less likely to enter a wrinkle both because of its thickness and its ability to compress in an area of stress 45 rather than folding on itself.

As may be recognized by those of ordinary skill in the pertinent art based on the teachings herein, numerous modifications, changes and/or additions may be made to the above-described and other embodiments of the present invention without departing from the scope of the invention as defined in the claims. For example, the imaging markers may include additional components or layers or fewer components or layers, may be made of any of numerous different materials, and/or may take any of numerous different shapes and/or configurations, that are currently known or that later become known. In addition, the shapes and/or configurations of the marker portion and/or spacer, and/or their materials of construction, may be changed as required to work with different types of imagers, imaging processes, or imaging systems, that are currently known or that later become known, including without limitation, x-ray, CT, Digital Breast Tomosynthesis, PET, MRI, or NMR imaging processes and systems. Accordingly, the configurations and/or materials of the components may be selected as dictated by the particular imager, imaging process, or imaging system with which the imaging marker is to be used, so that each component is radiopaque, is partially radiopaque, is partially radiolucent or partially radiotransparent (i.e., see through but visible), or is radiolucent or radiotransparent (i.e., invisible) on the image, as disclosed herein. Accordingly, this detailed description of embodiments is to be taken in an illustrative, as opposed to a limiting sense.

What is claimed is:

1. An imaging marker for use in connection with an imager, comprising:
a linear-shaped marker that is visible on an image of the marker taken by the imager in connection with an imaging procedure, wherein the linear-shaped marker is flexible and defines an underside and an elongated axis;
an adhesive; and
a foam spacer that is translucent or substantially invisible on the image of the marker taken by the imager in connection with the imaging procedure, wherein the foam spacer is located between the adhesive and the marker, and defines a thickness between the adhesive and substantially the entirety of the underside of the marker, the adhesive is configured to releasably attach the imaging marker to a surface of a person's skin undergoing the imaging procedure at an interface of the imaging marker and the skin, and the foam spacer spaces substantially the entirety of the underside of the marker away from the skin, wherein the foam spacer defines an axially-elongated portion extending along the elongated axis of the linear marker between the linear marker and the adhesive, and a plurality of laterally-extending portions, wherein a plurality of the laterally-extending portions are located on opposite sides of the elongated axis relative to each other, at least a portion of a plurality of the laterally-extending portions are axially spaced relative to each other along the elongated axis, and the foam spacer is configured to flex at least between the axially-spaced, laterally-extending portions to thereby allow the spacer to flex with the linear-shaped marker.

2. An imaging marker as defined in claim 1, wherein the foam spacer defines a plurality of pairs of laterally-extending portions extending laterally on opposite sides of the elongated axis relative to each other, and relatively narrow-width portions located between axially-spaced pairs of laterally-extending portions.

3. An imaging marker as defined in claim 1, wherein the imager generates images by transmitting radiation, the marker is formed by at least one radiopaque portion that is at least partially radiopaque, partially radiolucent at a level of radiation used by the imager in connection with the imaging procedure, and the foam spacer is substantially radiolucent at the level of radiation used by the imager in connection with the imaging procedure.

4. An imaging marker as defined in claim 1, wherein the foam spacer extends between the linear-shaped marker and the adhesive, and defines a thickness between an underside of the linear-shaped marker and the adhesive of at least about ½ millimeter.

5. An imaging marker as defined in claim 4, wherein the foam spacer defines a thickness between the underside of the linear-shaped marker and the adhesive within the range of about ½ millimeter to about 1 millimeter.

6. An imaging marker as defined in claim 1, wherein the thickness between the underside of the linear-shaped marker and the adhesive is substantially uniform.

7. An imaging marker as defined in claim 1, wherein the adhesive defines an adhesive coating underlying the foam spacer.

8. An imaging marker as defined in claim 1, wherein the imaging marker is mounted on a releasable liner, and the releasable liner is releasably attached to the adhesive.

9. An imaging marker as defined in claim 8, wherein the marker defines a continuous linear shape extending along the elongated axis, and the releasable liner defines an axially-elongated shape extending along the elongated axis of the linear marker.

10. An imaging marker as defined in claim 9, wherein the linear marker and releasable backing are configured to be torn, cut, or separated at desired locations to form individual imaging markers therefrom at desired lengths.

11. An imaging marker as defined in claim 1, wherein the foam of the foam spacer is a thermoplastic or thermoset foam.

12. An imaging marker as defined in claim 11, wherein the foam of the foam spacer is a closed-cell foam.

13. An imaging marker as defined in claim 1, wherein the linear-shaped marker is made of a thermoplastic including a filler of sufficient density to make the marker visible on an image of the marker taken by the imager in connection with the imaging procedure.

14. An imaging marker for use in connection with an imager, comprising:
first means for releasably attaching the imaging marker to a surface of the skin of a person undergoing the imaging procedure, wherein the surface of the skin defines a curvilinear contour and a scar or other anatomical feature also defining a curvilinear contour;
second means visible on an image of the marker taken by the imager in connection with an imaging procedure and forming a line-shaped image thereof in the image, wherein the second means is for flexing and substantially conforming to the curvilinear contour of the skin and for following the curvilinear contour of the scar or other anatomical feature on the skin; and
third means located between the first and second means that is translucent or substantially invisible on the image of the marker taken by the imager in connection with the imaging procedure for spacing substantially the entirety of the underside of the first means away from the skin, for flexing and substantially conforming to the curvilinear contour of the skin and following the curvilinear contour of the scar or other anatomical feature on the skin, and for preventing forces exerted thereon in flexibly conforming to and following the curvilinear counters from detaching the first means from the skin.

15. An imaging marker as defined in claim 14, wherein the first means is an adhesive, the second means is an axially-elongated, line-shaped marker, and the third means is a foam spacer, wherein the foam spacer defines an axially-elongated portion extending along the elongated axis of the line-shaped marker between the line-shaped marker and the adhesive, and a plurality of laterally-extending portions located on opposite sides of the elongated axis relative to each other and axially-spaced relative to each other along the elongated axis to thereby define bridges located between laterally-extending portions.

16. An imaging marker as defined in claim 15, wherein the marker is made of a thermoplastic including a filler of sufficient density to make the marker visible on the image of the marker taken by the imager in connection with the imaging procedure.

17. A method comprising:
preparing an imaging marker for attachment to a surface of the skin of a subject to be imaged, wherein the surface of the skin defines a curvilinear contour and a scar or other anatomical feature also defining a curvilinear contour, the imaging marker includes a flexible line-shaped marker portion that is visible on an image of the marker taken by an imager in connection with an imaging procedure, an adhesive, and a foam spacer located between the line-shaped marker portion and the adhesive that is translucent or substantially invisible on the image of the marker taken by the imager in connection with the imaging procedure, and the preparing includes flexing and substantially conforming the line-shaped marker portion to the curvilinear contour of the skin and causing the line-shaped marker portion to substantially follow the curvilinear contour of the scar or other anatomical feature on the skin;
releasably attaching to the surface of the skin the imaging marker, and in connection with such attaching, substantially conforming the line-shaped marker portion to the curvilinear contour of the skin, and causing the line-shaped marker portion to substantially follow the curvilinear contour of the scar or other anatomical feature on the skin;
imaging with the imager the marker portion of the imaging marker and at least the portion of the person underlying the marker such that the marker portion is visible and the foam spacer is translucent or substantially invisible on the image of the marker taken by the imager; and
during the releasably attaching and imaging of steps (ii) and (iii), absorbing forces exerted on the imaging marker during the flexible conforming to and following of the curvilinear counters within the foam spacer, and thereby preventing the imaging marker from detaching from the skin during the releasably attaching and imaging of steps (ii) and (iii).

18. A method as defined in claim 17, wherein the imaging includes transmitting radiation through the imaging marker at a level at which the marker portion is partially radiopaque, partially radiolucent to the transmitted radiation and the spacer is substantially radiolucent to the transmitted radiation.

19. A method as defined in claim 17, wherein the scar or other anatomical feature defines a length, and method further comprising cutting the imaging marker to a length approximately equal to or greater than the length of the scar or other anatomical feature.
